(19) United States
(12) Patent Application Publication
(10) Pub. No.: US 2002/0143231 A1
(43) Pub. Date: Oct. 3, 2002

(54) Magnetic collar

(76) Inventor: Peter A. Kulish, Fountainville, PA (US)

Correspondence Address: PETER A. KULISH, 5525 SWAMP ROAD, FOUNTAINVILLE, PA, 18923, US

(21) Appl. No.: 09/962,420

(22) Filed: Sep. 25, 2001

Related U.S. Application Data
(60) Provisional application No. 60/219,505, filed on Jul. 20, 2000.

(51) Int. Cl. A61N 1/00

(52) U.S. Cl. 600/15

(57) ABSTRACT

A magnetic collar having a center of gravity as determined by the placement of a counterweight of sufficient mass as to orient the magnetic field of a permanent magnet in a pre-determined direction thus rendering magnetic therapy to specific area of the body.

### BACKGROUND OF THE INVENTION

This device relates to magnetic therapy apparatus as utilizing a controllable center of gravity to determine the orientation of a magnetic field emanating from a permanent magnet relative to a specific mass. Magnetotherapy has been known to ameliorate medical conditions in the body in a similar fashion as acupuncture pressure or acupuncture therapy, consequently proper orientation of the magnetic field relative to the subject being treated is of paramount importance in such therapy.

### SUMMARY OF THE INVENTION

For centuries, the use of acupuncture and similar therapies that rely on specific stimulation points of the body has been utilized to ameliorate or alleviate various medical conditions. With such therapies, proper location of the biological stimulus is of extreme importance

It is a goal of this invention to achieve the proper orientation of the magnetic field in a fashion that does not impart discomfort to the subject being treated. It is obvious that treatment devices that cause discomfort in their use are simply not worn or not worn for the prescribed treatment period.

In U.S. Pat. No. 3,921,620 a magnetic treatment device is depicted that uses the tension produced by a mechanical fastener as to produce a surface pressure condition as to maintain the orientation of the permanent magnet relative to the subject placed under treatment.

In such a situation, discomfort arises from the tension or pressure produced by the wearer's body and in some cases skin chaffing can result. In the current invention under discussion, a mechanical collar fastener still can be utilized, however, mechanical pressure is not required to orientate the permanent magnet. Rather, a counter weight that seeks its orientation with respect to center of gravity of the collar is utilized. It can be readily ascertained in this situation no pressure is placed on the subject in order to achieve orientation of the permanent magnet.

While a counter weight itself can be utilized as to generate the desired orientation, a fastener such as a buckle of sufficient mass can be utilized to orientate the permanent magnet. In such a situation, a buckle itself, providing it is of greater mass than the magnet, serves as the orientating means.

It should be noted in certain applications where the magnetic treatment points or area is in the lower portion of the subject, the magnet itself may serve as its own counterweight.

Also, it is another object of this invention that such a collar be utilized as a magnetic therapy device by pets and animals such as cats, dogs, or all other accessible animals such as sheep, cows, or zoological animals.

With respect to such animals, the orientation of the spine resides in a horizontal plane; hence, a collar or belt placed upon such subject undergoing treatment can readily take advantage of utilizing a device where the placement of the magnet is disposed relative to a counterweight or stabilizing positioning mass.

One of the operant factors in controlling pain of a subject involves the Hall Effect. In the Hall Effect, the passage of an electrical current through a magnet is displaced 90 arcuate degrees with respect to the magnetic field.

The redirection of neural currents by the Hall Effect is one of the main principles behind pain amelioration being produced in patients undergoing magnetic therapy.

Reduction of swelling, inflammation, and endema by magnetic stimulation are also other significant factors in pain relief. However, the reduction of swelling and inflammation is more directly related to forcing, by magnetic electromotive field, the site into the negatively charged healing function (i.e., Robert Becker, The Body Electric, 1985.)

The Hall Effect is the response of the electron with respect to an orthogonally applied magnetic field. In such a situation, the electron basically follows Faraday's motor rule and is deflected ninety arcuate degrees with respect to the incident magnetic field.

This is of significance, since the deflection of electrons flowing in nerve circuits can then be ameliorated through the application of a magnetic field.

The transverse flow of pain impulse can be diffused by the proper cellular potentiation, which reorganizes the traumatized cellular stasis to a healing, non-painful stasis.

One of the most important double-blind studies in pain control was conducted on a group of post-polio patients.

The research document: Response of Pain to Static Magnetic Fields in Post-Polio Patients” by Vallbona, Hazlewood, and Jurdia of the Department of Family and Community Medicine, Baylor College, Houston, and featured in the journal: Arch Physics Med. Rehabil. 1997 Nov., 78(11):1200 003.

The objective of the study was to determine if the chronic pain frequently presented by postpolio patients could be relieved by application of magnetic fields applied directly over an identified pain trigger point. The design of the study was a double randomized clinical trial. It was set in the postpolio clinic of a large rehabilitation hospital. Fifty patients with diagnosed postpolio syndrome who reported muscular or arthritic-like pain were treated with the application of an active or placebo 300 to 500 Gauss magnetic device to the affected area for 45 minutes. The patients who received the active device reported an average pain score decrease of 4.4±3.1 (p<0.0001) on a ten point scale. Those with the placebo devices experienced a decrease of 1.1±1.6 points (p>0.005). The proportion of patients in the active-device group who reported a pain score decrease greater than the average placebo effect was 76%, compared with a reported 19% from the placebo group (p<0.0001). In conclusion, the application of a device delivering static magnetic fields of 300 to 500 Gauss over a pain trigger point results in significant and prompt relief of pain in postpolio subjects.

The CVS magnetic placement location is a specific direct application for immediately electromotively stimulating and balancing the neurotransmitter chemistry equally throughout all the brain meridians. By the normal physical law functions of increased and equalized cellular electromotive potential, this transmits the neurotransmission signal more efficiently which aids in brain function and its increased coefficient response with the nervous system of the body aiding in a higher general vitality. This supports increased immune system functions, and the CVS placement stimulates and balances the blood chemistry for increasing cellular metabolic functions. It is a specific safe location for all horizontally mobile animals, unlike the primate, which can use the magnetic placement over the heart where it naturally and gravitationally remains in place due to the vertical posture of the primate. The heart location is not available because it can be a dangerous placement for the non-primate animal due to the possibility of the wrong magnetic field, which can readily and accidentally flow into the body due to its non-vertical posture from normal activity, which would be detrimental to the proper energy flows of the body, which is the essence of this invention for the safe CVS location.

### DRAWINGS OF PREFERRED EMBODIMENT

FIG. 1 is an orthogonal view of a preferred embodiment relating to the use of a solitary, unipolar permanent magnet;

FIG. 2 represents an orthogonal view of a preferred embodiment utilizing a plurality of unipolar permanent magnets.

FIG. 3 represents a means of retrofitting an existing strap or collar with a counterweight and permanent magnet.

### Reference Numerals

| | |
|---|---|
| 10 | strap |
| 20 | permanent magnet |
| 30 | counterweight |
| 40 | fastener |
| 50 | magnetic clip |
| 60 | counterweight clip |

### DESCRIPTION OF THE PREFERRED EMBODIMENT

In accordance with previous discussions, one of the functions of an applied magnetic field is the amelioration of pain through the use of the lateral displacements of electrons caused by the Hall effect, in which the flow of electrical impulses are shifted 90 arcuate degrees with respect to an orthogonal magnetic field.

As previously cited, this is merely one of the many effects that arise upon the application of a magnetic field to the living tissue.

Consequently, since the Hall Effect is a direct derivation of Faraday's Right Hand Motor Rule—one solitary pole of 2 magnets can be used for such therapy, since meaningful deflection of a pain impulse from the normal path to the brain can only be achieved in such manner. A bi-polar, or the simultaneous application of a N-S magnet, only causes the electric impulse or pain signal to zigzag and consequently be allowed to return to its normal path.

In order to fabricate a useful medical magnet therapy device the solitary or monopole face of a permanent magnet must be utilized.

Meeting such a requirement can be achieved through the use of a single permanent magnet or by 2 clusters or groups of permanent magnets configured in such orientation as to expose a predetermined pole of the magnet(s) to the tissue or region under treatment.

While there are advantages to using a relatively large sized magnet in magnetotherapy, it is also desirable to utilize a plurality of permanent magnets mounted to a flexible material to allow the magnets to conform to the surface area under treatment.

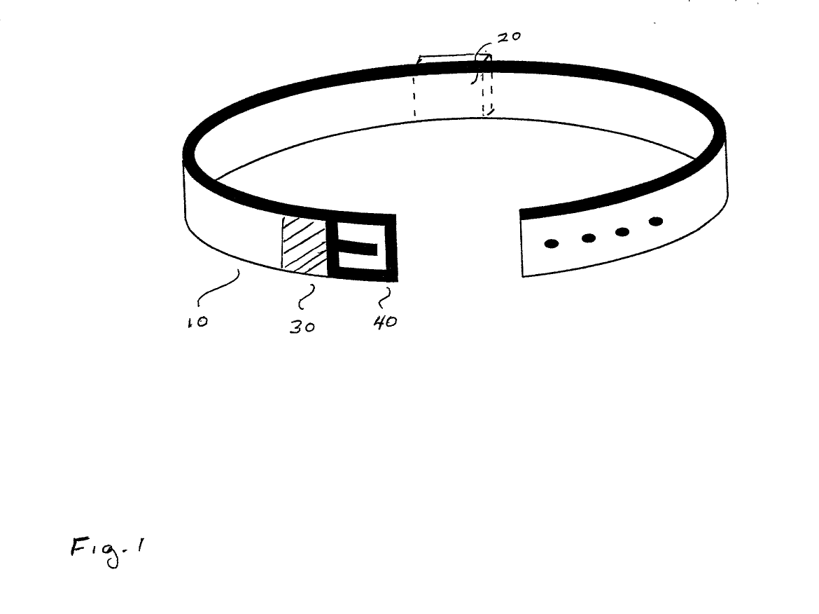

In accordance with the teachings as related above, FIG. 1 depicts a magnetic therapy collar comprising of a flexible strap (10) with permanent magnet (20) suitably attached to the flexible strap (10). The solitary permanent magnet has a predetermined face of the magnet (20) attached to strap (10) as to present the desired pole to the tissue or area to be treated.

A counterweight of sufficient mass (30) as to provide orientation of permanent magnet (20) is suitably attached to strap (10).

A magnetic device in accordance with the teachings of the device as depicted in FIG. 1 in which a solitary or plurality of magnets are attached to the flexible strap (10) and whose strength can be increased by the addition of like permanent magnets (20) and secured through magnetic means.

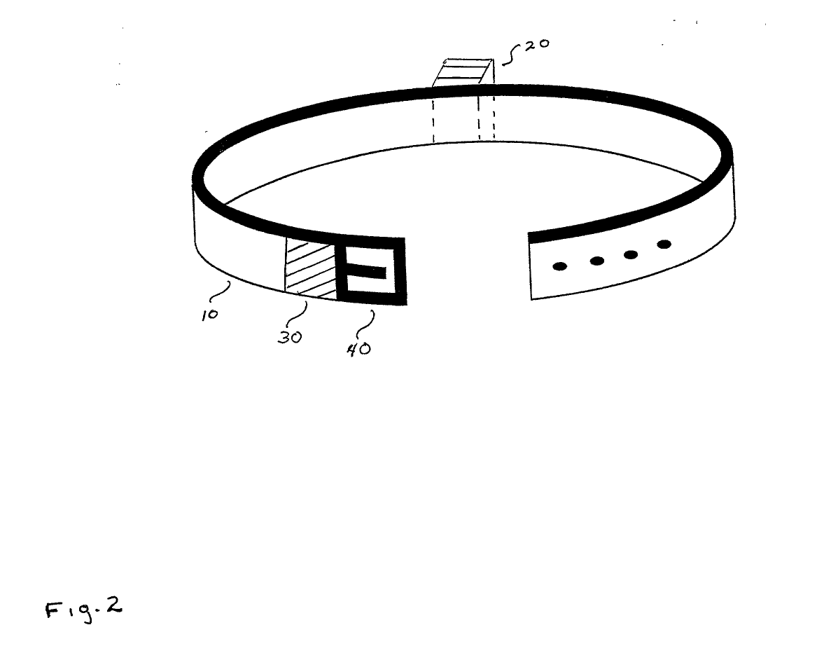

In FIG. 2, the flexible strap (10) consists of a tubular material, and consequently a permanent magnet placed within the tubular structure is free to longitudinally move within the structure. A permanent magnet (20) in place on the exterior surface of the tubular strap and through the friction and magnetic attraction between the magnets become attached to a desired position on the strap (10).

Similarly, additional magnets (20) can be placed over the existing magnet (20) residing within the tubular strap. The stacking of additional magnets (20) increases the effect power and penetration depth of the magnets utilized for therapeutic purposes.

With respect to FIGS. 1 and 2, a fastener (40) is used to secure the ends of strap (10) in such a asliion as to render it useable as a collar. Such fasteners such as, but not limited to, are reflected in the following examples: buckle, hook and eyelet, Velcro-type, magnetic and polymeric means.

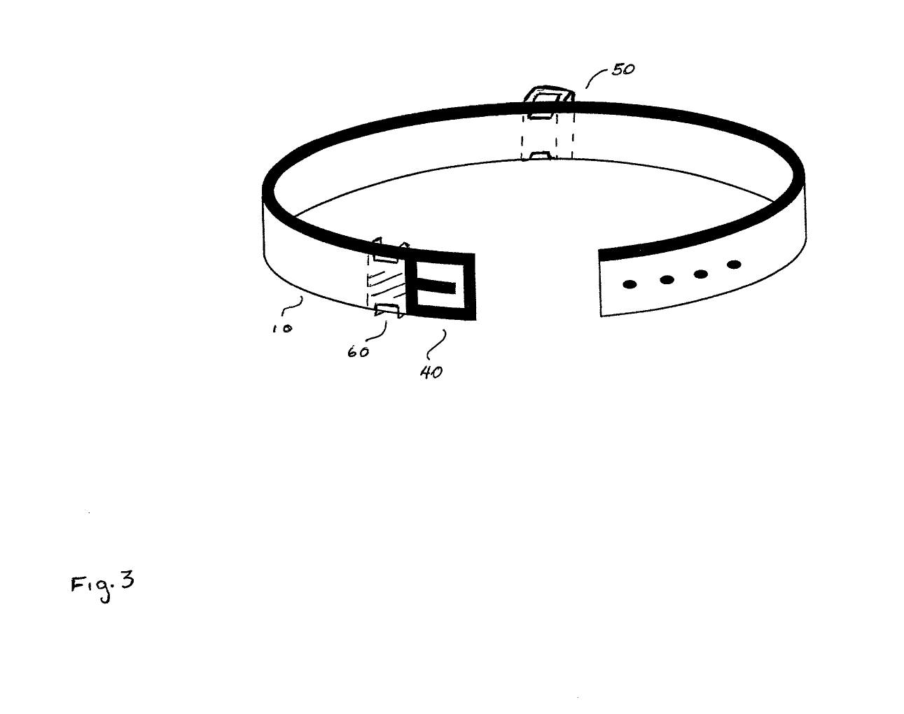

In FIG. 3 a magnet is secured via a clip (50) to an existing strap (10) or collar and similarly a counterweight (60) is secured via the clip (50) to an existing strap or collar. This allows component retrofitting the therapy device to an existing collar or strap.

What is claimed is:

1. A therapeutic device comprising of a permanent, strap, counterweight and fastener.

2. A device in accordance with claim 1 in which the magnet is a permanent magnet.

3. A device in accordance with claim 2 in which a solitary pole of a permanent magnet is exposed to the surface desired to be treated.

4. A therapeutic device comprising a series of permanent magnets, strap, counterweight and fastener.

5. A therapeutic device in accordance with claim 2 in which the series of stacked magnets, that can be of the following variety: ceramic, neodymium, samarium, cobalt, rare-earth element composition and compounds related thereto.

6. A therapeutic device consisting of an attachable magnet and an attachable counterweight as to allow the retrofitting of an existing strap or collar.
